(19) United States
(12) Patent Application Publication
(10) Pub. No.: US 2020/0022566 A1
(43) Pub. Date: Jan. 23, 2020

(54) ENDOSCOPIC APPARATUS

(71) Applicant: Leah Ruth Gonzalez, Tampa, FL (US)

(72) Inventor: Leah Ruth Gonzalez, Tampa, FL (US)

(21) Appl. No.: 16/039,449

(22) Filed: Jul. 19, 2018

(51) Int. Cl. A61B 1/00; A61B 1/31; A61B 1/015; A61B 1/06

(52) U.S. Cl. CPC A61B 1/00119; A61B 1/31; A61B 1/015; A61B 1/0676; A61B 1/00154; A61B 1/00082; A61B 1/00091

(57) ABSTRACT

The present invention discloses an endoscopic apparatus having a conduit-like overtube configured to receive an endoscope. Input openings at a proximal end of the overtube allow for fluid to be input therethrough. The fluid is expelled through output opening positioned along the length of the overtube. An annular balloon is positioned along the length of the overtube to inhibit movement and the removal of the overtube until the procedure is concluded.

### TECHNICAL FIELD

The present invention generally relates to an endoscopic apparatus, and more specifically, an endoscopic apparatus to be used during a colonoscopy screening and decompression procedure.

### BACKGROUND

Many endoscopic devices are used in a variety of medical procedures. Typically, these devices are utilized to enter and visualize the abdominal area via the oral or parenteral routes. Endoscopy has become increasingly useful in a variety of diagnostic and treatment procedures such as colonoscopies and colon decompression procedures. Colonoscopy is a procedure that enables an examiner (usually a gastroenterologist) to evaluate the inside of the colon. The colonoscope is comprised of a four-foot flexible tube having a camera and light source at the distal end. The procedure is performed as part of a screening program for colon cancer in addition to investigating blood in the stool, abdominal pain, diarrhea, or changes in bowel habits.

Patient comfort and safety continue to be a primary concern during colonoscopy exams. Passage of the colonoscope to the colon can be a source of discomfort for the patient and a significant hurdle to overcome for practitioners. The discomfort often leads to increased anesthesia administered to the patient which in-turn increases the danger and lengthens recovery times post-operation.

In current protocols, the colonoscope is suitably positioned near the target anatomy by inserting the device into the body cavity. Often, drainage of the colon is required due to blockage as a result of inflammation. A small tube within the inner channel of the scope facilitates the decompression of the colon. However, this tube is often too small in diameter to accomplish the decompression without getting bent, clogged, or dislodged.

It can be seen that an improvement in the arts related to endoscopic devices facilitating colon decompression is needed. Once such improvement is provided in the various embodiments described herein.

### SUMMARY OF THE INVENTION

This summary is provided to introduce a variety of concepts in a simplified form that are further disclosed in the detailed description of the invention. This summary is not intended to identify key or essential inventive concepts of the claimed subject matter, nor is it intended for determining the scope of the claimed subject matter.

The present embodiments disclose an appratus for colon screening and decompression. The apparatus is comprised of a conduit-like overtube including proximal and distal ends. The overtube is configured to receive an endoscope via an input port positioned at the proximal end. A fastening means, such as a snap-fit mechanism, releasably engages the overtube with the endoscope during scanning of the target anatomy. At least one input opening at the proximal end of the overtube receives water or likewise fluid therethrough. The input opening is in fluid communication with a plurality of output openings along the length of the overtube. An annular balloon is positioned along the length of the overtube to inhibit the removal of the overtube

In one embodiment, the endoscope is defined as a colonoscope having at least one optical component, and a light source positioned the distal end thereof. The overtube is configured to not obstruct the view of the optical component during the screening of the target anatomy within the body cavity.

In another embodiment, the apparatus includes a reservoir for the collection of fluids and solids flushed or otherwise removed from the target anatomy.

A method of using the apparatus includes the steps of at least one medical practitioner performing the steps of lubricating the overtube and positioning the endoscope within the overtube before the insertion of the apparatus within the body cavity. The endoscope is positioned such that the optical component can suitably analyze the target anatomy during a screening procedure. The annular balloon is inflated to inhibit the movement of the overtube and endoscope throughout screening and flushing. Once the target anatomy has been screened, the endoscope is disengaged, using the fastening means, and removed from within the overtube. The overtube is removed following the flushing and decompression of the body cavity.

In one embodiment, the overtube is an elongated member having a length suitable to view the target anatomy via at least one optical component and at least one light source positioned at the distal end of the endoscope.

Moreover, in accordance with a preferred embodiment of the present invention, other aspects, advantages, and novel features of the present invention will become apparent from the following detailed description in conjunction with the drawings.

### BRIEF DESCRIPTION OF THE DRAWINGS

A more complete understanding of the present invention, and the advantages and features thereof, will be more readily understood by reference to the following detailed description when considered in conjunction with the accompanying drawings wherein:

FIG. 1 illustrates a front elevation view of the endoscopic apparatus and overtube disposed within a body cavity, according to an embodiment of the present invention;

FIG. 2 illustrates a front elevation view of the overtube disposed within a body cavity having the endoscope removed therefrom, according to an embodiment of the present invention;

FIG. 3 illustrates a cross-sectional view of the overtube and endoscopic apparatus, according to an embodiment of the present invention; and

FIG. 4 illustrates a flowchart of a method of using the endoscopic apparatus, according to an embodiment of the present invention.

### DETAILED DESCRIPTION

The specific details of the single embodiment or variety of embodiments described herein are to the described apparatus and method of use. Any specific details of the embodiments are used for demonstration purposes only and not unnecessary limitations or inferences are to be understood therefrom.

Any reference to “invention” within this document is a reference to an embodiment of a family of inventions, with no single embodiment including features that are necessarily included in all embodiments, unless otherwise stated. Furthermore, although there may be references to “advantage's” provided by some embodiments, other embodiments may not include those same advantages, or may include different advantages. Any advantages described herein are not to be construed as limiting to any of the claims.

Before describing in detail exemplary embodiments, it is noted that the embodiments reside primarily in combinations of components related to the apparatus. Accordingly, the apparatus components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

As used herein, relational terms, such as “first” and “second” and the like, may be used solely to distinguish one entity or element from another entity or element without necessarily requiring or implying any physical or logical relationship or order between such entities or elements.

As used herein, the term endoscope relates to an instrument which can be introduced into the body to give a view of its internal parts. Commonly, these devices are comprised of a visual component and illuminated component positioned at a distal end of the device. Examples include colonoscopes, cystoscopes, nephroscopes, bronchoscopes, arthroscopes, and laparoscopes.

In general, the embodiments described herein relate to an endoscopic apparatus configured to facilitate, decompression of the colon. The apparatus is comprised of an overtube configured to receive a colonoscope or similar optical device employed in various endoscopic screening and analysis procedures. The overtube is a sleeve-like elongated member having an inner diameter larger than the endoscope to provide a conduit for passage of the endoscope into the digestive tract.

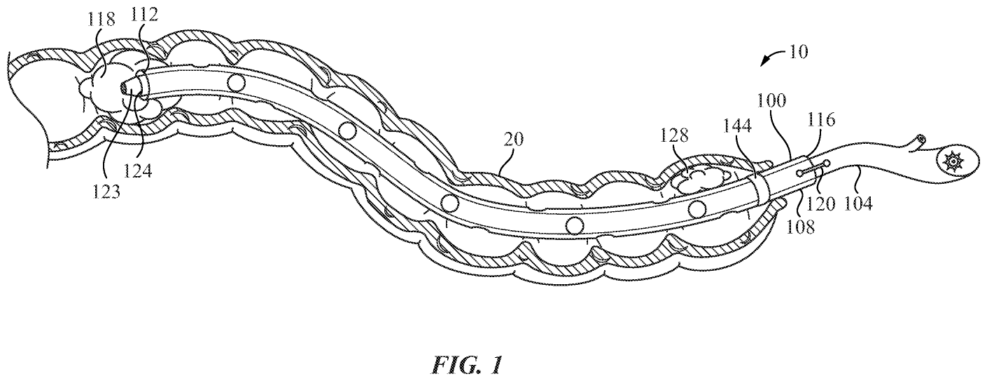

FIG. 1 illustrates the endoscopic apparatus 10 inserted within the body cavity 20 of a patient undergoing a colonoscopy-related procedure. The overtube 100 is positioned to provide a conduit for an endoscope 104 to pass therethrough. It is understood that one skilled in the arts may appreciate that the present invention can be utilized in a variety of procedures including gastroscopy, sigmoidoscopy and colonoscopy, esophagogastroduodenoscopy (EGD), endoscopic retrograde cholangiopancreatography (ERCP), and bronchoscopy.

The overtube 100 includes a proximal end 108 and a distal end 112. During a procedure, the proximal end 108 is inserted into the body cavity and guided manually by the medical practitioner to the target anatomy. An opening 116 at the proximal end 108 permits the disposal of an endoscopic unit (endoscope 104) therein. The distal end 112 is inserted orally or parenterally to the predetermined target anatomy 118 within the patient.

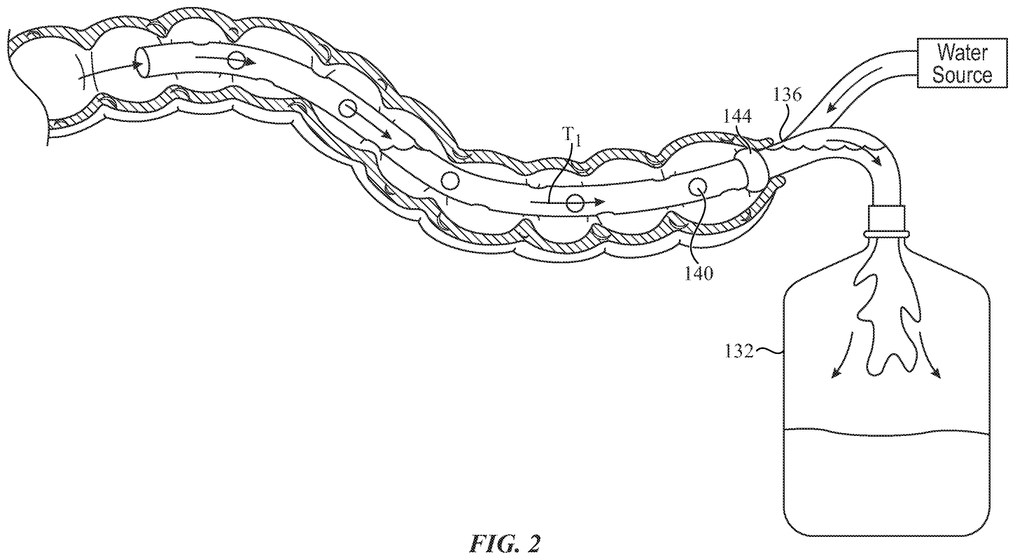

In a preferred embodiment, the overtube 100 includes fastening means 120 at the proximal end 108 to releasably engage with the proximal end of the endoscope 104. Fastening means may include a clip, snap-fit mechanism, strap, clasp, locking screw/bolt, friction fitting mechanism, or likewise component known in the arts. The fastening means should releasably engage without imparting undue discomfort to the patient. The fastening means 120 engages with a complementary receiving portion on the proximal end of the endoscope 104. Following the screening procedure, the endoscope can be disengaged from the overtube 100 and removed while leaving the overtube 100 in position at the target anatomy 118 as shown in FIG. 2. This action facilitates the egress of bodily fluids and solids 128 through the overtube 100 and into reservoir 132. In general body fluids and solids 128 will egress along travel path T₁.

In a preferred embodiment, a beveled portion 123 is provided at the distal end of the endoscope to facilitate insertion into the body cavity. The beveled portion 123 permits the distal end to have a smaller diameter to ease discomfort to the patient.

In a preferred embodiment, an input port 136 permits the ingress of fluid to aid in flushing the body cavity 20 of the patient. A plurality of openings 140 positioned along the length of the overtube 100 allow for the flushing fluid to egress therethrough. The openings allow for the overtube 100 to aid in flushing of the cavity of bodily fluids and solids 128 along travel path T₁, as commonly practiced and readily known in the arts. Flushing will be aided by the use of the overtube 100 in contrast to a small inner channel currently used.

Following the removal of the endoscope 104, suction can be applied to the overtube 100 to facilitate the removal of bodily fluids and solids.

In one embodiment, an inflatable balloon 144 is positioned along the length of the overtube 100 such that the balloon aids in keeping the apparatus 10 in an optimal position within the cavity 20. Typically, the balloon 144 is an annular balloon encircling the overtube 100 near the proximal end 108. In the illustrated example, the balloon 144 is inflated within the rectum of the patient to maintain the position of the apparatus 10.

In an alternate example, wherein EGD procedure is performed, the inflatable balloon 144 functions to dilate a strictured esophagus. In many procedures, the interior of the balloon 144 may be utilized to inflate and observe the cavity using fluoroscopy, x-ray, or other screening implements.

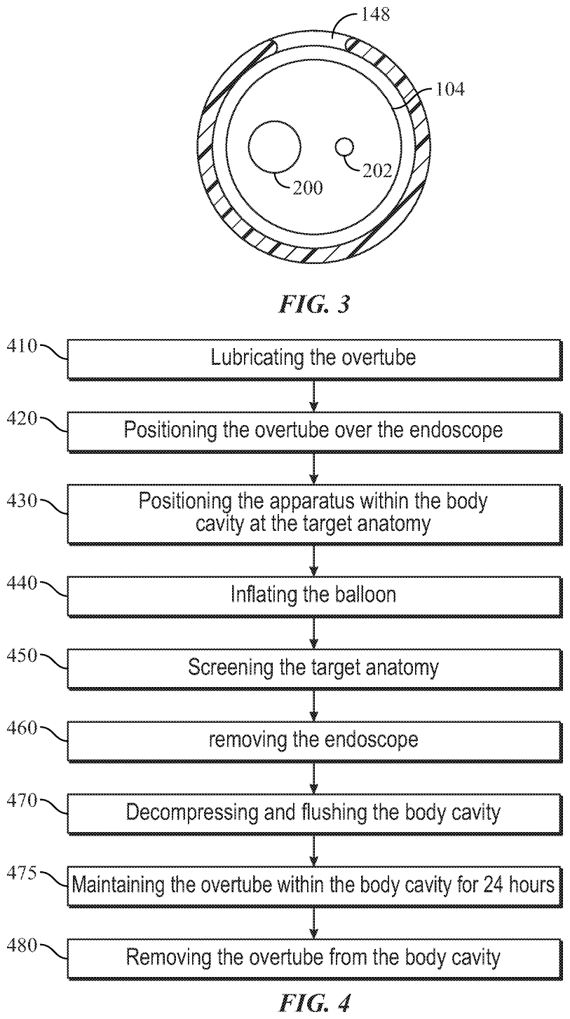

Now referring to FIG. 3, a cross-section of the apparatus 10 is shown having an optical component 200 and light source 202 positioned at the distal end 124 of the endoscope 104. A channel 148 is in fluid communication with input port 136 (shown in FIG. 2). Before the procedure, lubrication is applied between the inner surface of the overtube 100 and the outer surface of the endoscope 104. Lubrication reduces friction of the surfaces during the removal of the endoscope 104 while the overtube 100 remains in the body cavity 20. The lubrication will also reduce discomfort experienced by the patient, as well as reduce unwanted movement of the overtube 100.

In an embodiment, the endoscope is comprised of a plurality of control systems having controls thereon. The controls allow the medical practitioner to control and guide the endoscope and overtube through vessels and cavities of a patient using known means.

In an alternate embodiment, the overtube can include a proximal seal for the endoscope to prohibit unwanted movement or egress of fluids.

In order to decrease discomfort experienced during the procedure, dimensions of the overtube 100 can include an inner diameter of 11 mm, an outer diameter of 13.3 mm, and a length of 1828 mm. The inner diameter facilitates the flow of fluids and solids 128 therethrough while the outer diameter remains small enough to minimize pain due to the insertion retainment of the overtube 100 within the cavity 20. It is understood that dimensions are provided by way of example of a useful embodiment. A variety of dimensions may be utilized in practice dependent on the procedure type.

In a preferred embodiment, the overtube 100 is transparent or semitransparent with X-ray markers on the exterior surface to aid in visualization of the overtube 100 when used to dilate strictures. This permits the visualization of the overtube 100 and bodily fluids therein when accessing blood flow.

As noted, it is contemplated the overtube can be constructed from a variety of suitable materials. These suitable polymeric materials include but are not limited to polytetrafluoroethyl one (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®, ethylene vinyl acetate copolymers (EVA), silicones, polyethylene (PE), Marlex high-densitypolyethylene, Marlex low-density polyethylene, linear low density polyethylene (for example REXELL®, polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), polyparaphenylene terephthalamide (for example, KEVLAR®, polysulfone, nylon, nylon 12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styfene) (for example, SIBS and/or SIBS 50A), polycarbonates, ionomers, biocompatible polymers, other suitable materials, or mixtures, combinations, copolymers thereof, polymer/metal composites, and the like.

Now referring to FIG. 4, a method is shown for performing an endoscopic procedure using the apparatus 10. Each step is preferentially performed by a qualified medical practitioner in a controlled environment. In step 410, lubrication is applied to a plurality of surfaces on the apparatus. This can include the outer surface of the overtube, the inner surface of the overtube, and the outer surface of the endoscope. In step 420, the overtube is positioned over the endoscope prior to the insertion of the apparatus (step 430) within the body cavity such that the optical components can screen the target anatomy. The endoscope can be removably engaged with the overtube using fastening means such as a snap-fit mechanism. In step 440, the balloon is inflated to retain the apparatus at the desired position to screen the target anatomy in step 450.

Following the screening of the target anatomy, in step 460 the endoscope can be removed by disengaging the fastening means and pulling the endoscope from the overtube. The removal is facilitated by the lubrication applied in step 410. In step 470, the body cavity is decompressed and flushed to remove undesired bodily fluids and solids. It is understood that step 470 can be omitted during a procedure where it is unnecessary or unpractical. Optionally, in step 475, the overtube can be retained within the body cavity for a time period, such as a 24 hours. During this time period, flushing or draining can continue to occur depending on direction from the medical practitioner. In step 480, the overtube is removed from the body cavity to conclude the procedure.

As described above, the method can result in a drastic reduction in procedure time in addition to the decreased use of anesthesia. The apparatus, along with the method of use is intended to result in decreased discomfort throughout an otherwise painful procedure.

Many different embodiments have been disclosed herein, in connection with the above description and the drawings. It will be understood that it would be unduly repetitious and obfuscating to literally describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

An equivalent substitution of two or more elements can be made for any one of the elements in the claims below or that a single element can be substituted for two or more elements in a claim. Although elements can be described above as acting in certain combinations and even initially claimed as such, it is to be expressly understood that one or more elements from a claimed combination can in some cases be excised from the combination and that the claimed combination can be directed to a subcombination or variation of a subcombination.

It will be appreciated by persons skilled in the art that the present embodiment is not limited to what has been particularly shown and described hereinabove. A variety of modifications and variations are possible in light of the above teachings without departing from the following claims.

What is claimed is:

1. An endoscopic apparatus for colon screening and decompression comprising:
a. a conduit-like overtube including a proximal end and a distal end, the overtube configured to receive an endoscope via an input port at the proximal end of the overtube;
b. a fastening means configured to releasably engage the overtube and the endoscope;
c. at least one input opening at the proximal end of the overtube in fluid communication with a plurality of output openings along the length of the overtube, wherein the at least one input opening and the plurality of output openings are configured to facilitate ingress and egress of fluids therethrough during flushing of a cavity; and
d. an annular balloon positioned along the length of the overtube, wherein the annular balloon is configured to inhibit removal of the overtube.

2. The apparatus of claim 1, wherein the endoscope is a colonoscope having an optical component and a light source positioned at the distal end of the colonoscope.

3. The apparatus of claim 2, wherein the overtube does not obstruct the optical component and light source.

4. The apparatus of claim 2, wherein the colonoscope is positioned at a target anatomy within a body cavity.

5. The apparatus of claim 4, wherein the overtube facilitates the removal of a fluid or solid to decompress the colon.

6. The apparatus of claim 5, including a reservoir positioned at the proximal end of the overtube, the reservoir is configured to receive waste from the patient.

7. The apparatus of claim 2, wherein the fastening means is a snap-fit mechanism permitting the releasable engagement and removal of the colonoscope from within the overtube.

8. An endoscopic system comprising:
a. a conduit-like overtube including a proximal end and a distal end, the overtube configured to receive an endoscope via an input port at the proximal end of the overtube;
b. a fastening means positioned at the distal end of the overtube, the fastening means configured to releasably engage with a distal end of the endoscope;
c. at least one input opening at the proximal end of the overtube in fluid communication with a plurality of output openings along the length of the overtube, wherein the at least one input opening and the plurality of output openings are configured to facilitate ingress and egress of fluids therethrough during flushing of a cavity; and
d. an annular balloon positioned along the length of the overtube, wherein the annular balloon is configured to inhibit removal of the overtube.
e. at least one medical practitioner performing the steps of:
i. lubricating the overtube;
ii. positioning the endoscope within the overtube;
iii. positioning the distal ends of the overtube and endoscope at the target anatomy within the cavity;
iv. inflating the annular balloon;
v. screening the target anatomy;
vi. disengaging and removing the endoscope from within the overtube; and
vii. removing the overtube from the cavity.

9. The System of claim 8, further comprising the step of decompressing the colon following the removal of the endoscope from within the overtube.

10. The system of claim 9, further comprising the step of forcing fluid through the at least one input opening to flush the colon.

11. The system of claim 10, wherein a channel facilitates the flow of fluid from the at least one input opening to the plurality of output openings.

12. The apparatus of claim 9, including a reservoir positioned at the proximal end of the overtube.

13. The system of claim 12, further comprising the step of collecting waste in the reservoir.

14. The system of claim 8, wherein lubrication is applied to at least two of the following;
an outer surface of the overtube, an inner surface of the overtube, and the outer surface of the endoscope.

15. A colon decompression system comprising:
a. a conduit-like overtube including a proximal end and a distal end, the overtube configured to receive an endoscope via an input port at the proximal end of the overtube;
b. a fastening means positioned at the distal end of the overtube, the fastening means configured to releasably engage with a distal end of the endoscope;
c. at least one input opening at the proximal end of the overtube in fluid communication with a plurality of output openings along the length of the overtube, wherein the at least one input opening and the plurality of output openings are configured to facilitate ingress and egress of fluids therethrough during flushing of a cavity; and
d. an annular balloon positioned along the length of the overtube, wherein the annular balloon is configured to inhibit removal of the overtube.
e. at least one medical practitioner performing the steps of:
i. lubricating the overtube;
ii. positioning the endoscope within the overtube;
iii. positioning the distal ends of the overtube and endoscope at the target anatomy within the cavity;
iv. inflating the annular balloon;
v. screening the target anatomy;
vi. disengaging and removing the endoscope from within the overtube;
vii. flushing the target anatomy;
viii. collecting waste in a reservoir, the reservoir positioned at the proximal end of the overtube; and
ix. removing the overtube from the cavity.

16. The system of claim 15, wherein the overtube is an elongated member having a length suitable to view the target anatomy via at least one optical component and at least one light source positioned at the distal end of the endoscope.

17. The system of claim 15, further comprising a snap-fit mechanism configured to releasably engage the endoscope with the overtube.

18. The system of claim 15, wherein lubrication is applied to at least two of the following; an outer surface of the overtube, an inner surface of the overtube, and the outer surface of the endoscope.

19. The system of claim 15, wherein the overtube is constructed of a semi-rigid material.

20. The system of claim 15, wherein an inner diameter of the overtube is at least 11 mm.
